(19) United States
(12) Patent Application Publication
(10) Pub. No.: US 2012/0037515 A1
(43) Pub. Date: Feb. 16, 2012

(54) IMPEDIMETRIC SENSORS USING DIELECTRIC NANOPARTICLES

(75) Inventor: Rajendra Solanki, Portland, OR (US)

(73) Assignee: TheStateof Oregonactingbyand throughthestateBoard ofHigherEducationon behalf of thePortlandstateUniv

(21) Appl. No.: 13/138,865

(22) PCT Filed: Apr. 14, 2010
(86) PCT No.: PCT/US2010/001112

§ 371 Date: Oct. 11, 2011

Related U.S. Application Data
(60) Provisional application No. 61/212,821, filed on Apr. 15, 2009.

(51) Int. Cl. G01N 33/50; C25B 11/00; G01N 27/26; B82Y 99/00; B82Y 35/00; B82Y 30/00; B82Y 5/00; B82Y 15/00

(52) U.S. Cl. 205/780.5; 205/789; 204/280; 977/773; 977/762; 977/742; 977/775; 977/832

(57) ABSTRACT

A method for electrochemical impedance spectroscopy uses interdigitated electrodes functionalized with a first species and nanoparticles functionalized with a second species that preferentially attaches to the first species. The nanoparticles are composed of a material with a dielectric constant (k value) greater than 2. The chemically functionalized electrodes are then exposed to a solution containing the chemically functionalized nanoparticles which then become immobilized on the electrodes through the attachment of the first species to the second species. The impedance spectrum is measured and an amount of the first species is then determined from the measured spectrum. Because the high-k dielectric nanoparticles increase the double-layer capacitive impedance, the sensitivity of determining the amount of the first species attached to the second species is enhanced.

### FIELD OF THE INVENTION

The present invention relates generally to impedimetric sensors and methods for impedance spectroscopy. More specifically, it relates to methods for electrochemical impedance spectroscopy.

### BACKGROUND OF THE INVENTION

Impedimetric sensors are used in impedance spectroscopy where changes in complex resistance (impedance) are measured as a function of frequency. The technique of impedance spectroscopy can be used for sensing physical, chemical, or biological species. See, for example, US Patent Application Publication 2008/0036471, which is incorporated herein by reference. In electrochemical impedimetric sensing, electrodes are brought into contact with a solution containing a species to be detected (i.e., an analyte). For selective detection, the electrodes can be chemically treated so that a specific analyte can be immobilized on the electrodes. AC voltages are then applied to a circuit containing the electrodes, and the resulting impedance is measured. The AC voltage is typically small (i.e., tens of mV) in order to minimize altering the properties of the analyte immobilized on the metal electrodes. The impedance (Z) may be measured by detecting the current produced in response to the applied voltage and its phase difference with respect to the applied voltage. The magnitude of the impedance is the ratio of the voltage and current amplitudes, while the phase of the impedance is the difference in phase between the voltage and current. This impedance can be represented as a sum of real component (Zᵣ) which is the purely resistive component and an imaginary component (Zᵢₘ) which is called the reactance. In general, the impedance depends on the frequency (ω) of the applied AC signal: i.e., Z(ω)=Zᵣ(ω)+iZᵢₘ(ω). Because the impedance of the electrodes will change due to the presence of the immobilized analyte, it is possible to detect the presence of analyte by impedance measurements. The magnitude of the impedance signal can be calibrated to determine the amount of analyte. Unfortunately, however, current impedimetric sensors and associated techniques have limitations in detection sensitivity. Consequently, the presence of analytes below a given concentration can not presently be detected. For example, some diseases such as HIV and certain kinds of cancer can not currently be detected at an early stage. Accordingly, it would be a significant advance in the art to enable the detection of such low-concentration analytes.

### SUMMARY OF THE INVENTION

According to one aspect, the present invention provides a method for electrochemical impedance spectroscopy. According to the method, interdigitated electrodes are chemically functionalized with a first species, and high-k dielectric nanoparticles are chemically functionalized with a second species that preferentially attaches to the first species. The first and second species may be biological species such as, for example, an antibody and complementary antigen or two complementary DNA strands. The dielectric nanoparticles are composed of a material with a static dielectric constant (k value) greater than 2. For greater sensitivity, the k value may be greater than 10. They may take various forms such as nanowires, nanotubes, nanorods, nanospheres, nanofibers, nanopowders, nanoclusters, nanocrystals, or nanobeads. The nanoparticles may be composed of an organic or inorganic material. The chemically functionalized interdigitated electrodes are then exposed to a solution containing the chemically functionalized nanoparticles which then are allowed to be immobilized on the electrodes through the attachment of the first species to the second species. The impedance values at a collection of distinct applied AC frequencies are then measured in a circuit that includes the electrodes. An amount of the first species is first detected and then quantified from the measured impedance values. Because the presence of dielectric nanoparticles change the double-layer capacitance, the sensitivity of determining the amount of the first species attached to the second species is improved.

Preferably, impedance values are measured by generating an AC signal at a predetermined frequency and applying the AC signal to the circuit. The frequency-dependent impedance of the circuit produced in response to the applied AC signal at the predetermined frequency is then measured. This is repeated with the predetermined frequency ranging over multiple distinct predetermined frequencies to obtain measured impedance values over a frequency range, e.g., 1 Hz to 100 kHz or more preferably 50 Hz to 50 kHz. The amount of the first species may then be determined by analyzing the measured impedance values to determine the net double-layer capacitance at the chemically functionalized interdigitated electrodes. The change in the capacitive impedance can be calibrated with respect to the amount of first species. Once calibrated, then the impedance measurements can be used to detect for the presence of that species and if present, can be used to determine its amount.

In another aspect, the invention provides a kit that includes a solution containing nanoparticles that are chemically functionalized with a first species and that have dielectric constants greater than 2. The kit also includes interdigitated electrodes that are capable of chemically functionalized with a second species that preferentially attaches to the first species. Alternatively the nanoparticles may be capable of being chemically functionalized with the first species; and the interdigitated electrodes are chemically functionalized with the second species. The kit may also include a buffer solution. The test analyte will first be mixed with the functionalized nanoparticles and then this sample will be exposed to the functionalized electrodes on the chip for testing.

### BRIEF DESCRIPTION OF THE DRAWINGS

FIG. 1 is an electrical schematic of an equivalent Randles circuit used to model an interdigitated electrode circuit according to an embodiment of the invention.

FIG. 2 is a Nyquist plot of impedance values for an interdigitated electrode circuit according to an embodiment of the invention.

FIG. 3 is a Nyquist plot of impedance values for an interdigitated electrode circuit showing the effect of various concentrations of KCl according to an embodiment of the invention.

FIG. 4 is a Nyquist plot of impedance values for an interdigitated electrode circuit showing the effect of antigen (Gliadin) immobilization and then antibody (Anti GP) binding on the electrode surface according to an embodiment of the invention.

FIG. 5 is a graph of impedance magnitude as a function of frequency for an interdigitated electrode circuit according to an embodiment of the invention, showing the component of the impedance that dominates at various frequencies.

FIG. 6A is a schematic diagram illustrating the process of functionalizing an interdigitated electrode with a first species, such as an antibody.

FIG. 6B is a schematic diagram illustrating the process of functionalizing a dielectric nanoparticle with a second species, such as an antigen.

FIG. 6C is a schematic diagram illustrating the process of immobilizing or attaching the functionalized nanoparticles of FIG. 6B to a functionalized interdigitated electrode of FIG. 6A.

FIG. 7A is a schematic diagram illustrating the process of functionalizing an interdigitated electrode with a first species, such as an antigen.

FIG. 7B is a schematic diagram illustrating the process of functionalizing a dielectric nanoparticle with a second species, such as an antibody.

FIG. 7C is a schematic diagram illustrating the process of immobilizing or attaching the functionalized nanoparticles of FIG. 7C to the functionalized interdigitated electrode of FIG. 7A.

FIG. 8 is a Nyquist plot of impedance values comparing different stages of biomolecular binding activity at an electrode surface according to an embodiment of the invention.

FIG. 9 is a schematic diagram illustrating a kit including interdigitated electrodes on a chip, a solution containing high-k nanoparticles, and a buffer solution, according to an embodiment of the invention.

### DETAILED DESCRIPTION

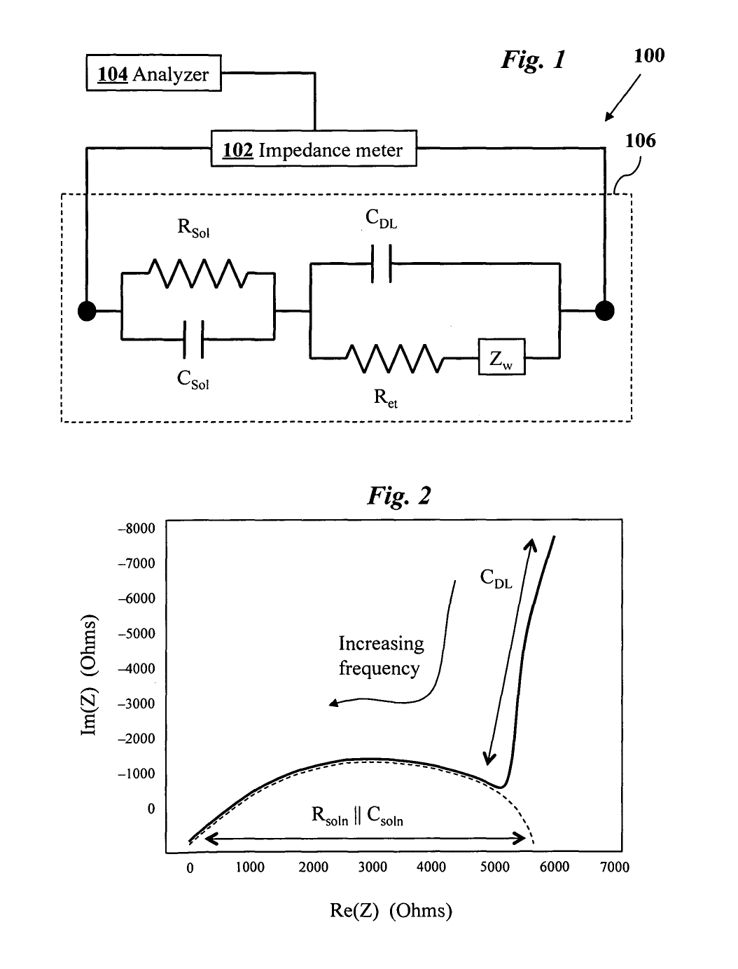

An impedimetric sensor 100 according to one embodiment includes interdigitated electrodes connected to an impedance meter 102 as shown in FIG. 1. For small applied voltages (i.e., tens of mV), an equivalent Randles circuit 106 may be used to model the circuit for the impedimetric sensor. Prior to the impedance measurement, an analyte is immobilized on the surfaces of the electrodes. During impedance measurement, the bare electrodes are exposed to a buffer solution having resistance R_{Sol} and an AC probe voltage V(t)=V₀+ΔV sin (2πft) is applied to the circuit. In response to the applied voltage, a double layer of charges is produced near the electrode surface, resulting in a double-layer capacitance C_{DL} which will depend upon the amount of analyte attached to the electrode surface as well as on the intermediate binding or self aligned molecules (SAM) and the analyte The circuit is also characterized by a Warburg impedance Z_{w}, which can arise from mass transfer, and electron transfer resistance Rₑₜ due to the immobilized analyte at the electrodes.

The impedance of the circuit is measured by the impedance meter 102 for applied AC voltages over a range of frequencies f to obtain an impedance spectrum which is analyzed by analyzer 104. Typically, the number of data points collected may range from 100 to 1000. The spectrum can be graphically represented in a Nyquist plot as shown in FIG. 2, where the vertical axis indicates the imaginary component of the impedance and the horizontal axis indicates the real component of the impedance. The Nyquist plot of the spectrum can be analyzed to determine circuit parameters. The spectrum generally has the shape of a straight line for the lower frequencies and a semi-circle for the higher frequencies. The diffusion corresponds to the low frequency vertical tail of the spectrum. The high frequency semi-circle portion of the spectrum corresponds to the parallel combination of double layer capacitance (C_{DL}), as well as the electron transfer resistance (Rₑₜ) and the resistance (R_{Sol}) of the solution. The width of the semicircle (at Zᵢₘ=0) is the electron transfer resistance (Rₑₜ).

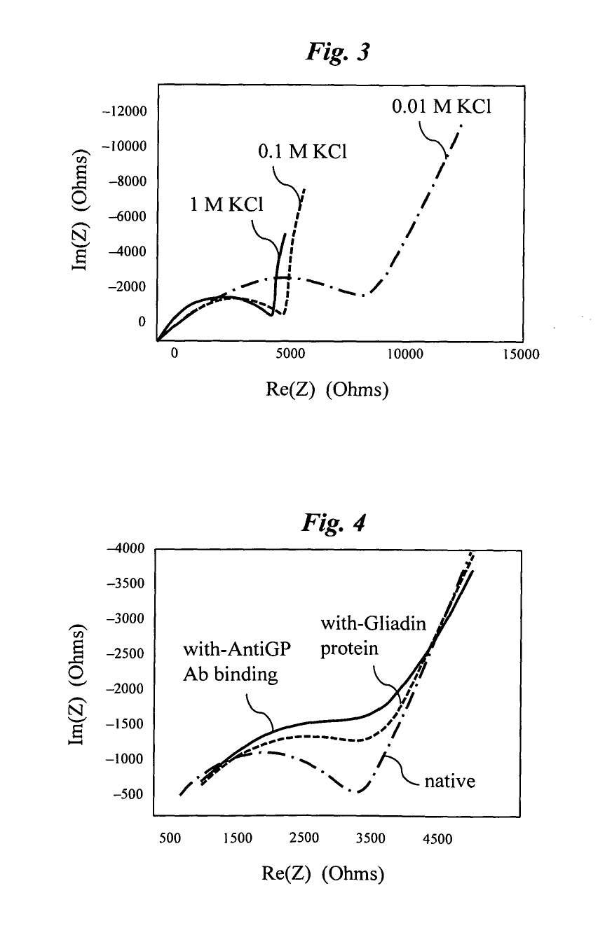

When analytes are immobilized on the electrodes, the circuit parameters are changed and, consequently, this alters the shape of the Nyquist plot (e.g., the diameter of the semicircle and other attributes). Thus, this change in the impedance spectrum can be used to detect the amount of analyte that has become immobilized. For example, Nyquist plots produced from impedance spectrum measurements under different conditions are shown in FIGS. 3 and 4. Three impedance spectra are shown in FIG. 3, where each spectrum corresponds to a different concentration of KCl in the solution. Changes in the concentration of KCl alter the diameter of the semicircle and the position and slope of the low frequency tail. FIG. 4 shows spectra that illustrate the effect of antigen immobilization and antibody binding on the electrode surface. One spectrum corresponds to the native (i.e., bare) electrode. A second spectrum shows the effect of gliadin protein (i.e., antigen) attached to the electrode, and a third spectrum shows the further effect of binding antiGP Ab (i.e., antibody) to the protein.

An important property of impedance spectroscopy is the sensitivity for the detection of analytes. Accordingly, various techniques have been developed previously to increase the sensitivity. For example, Au nanoparticles have been incorporated in impedance sensors to increase the effective surface area of the electrodes and improve the electrical connectivity through the nanoparticle network. As expected, the electron transfer resistance decreases with increase of the number of Au nanoparticle layers due to reduction in the conductivity. However, its value increases with binding of the electrically insulating human IgG, which is used as the detection signal.

Another approach conjugates Au nanoparticles with an analyte in the solution so that the impedance signal is amplified when the nanoparticles become embedded onto the surface of the sensing electrodes. Since the Au nanoparticles are embedded within the insulating analyte, the value of Rₑₜ is reduced. Since several analytes can be conjugated to each Au nanoparticle, the signal can be slightly amplified. Note that Au is an excellent conductor and is not a dielectric material. More generally, metallic nanoparticles will not contribute to the capacitance (impedance) of the double layers, and will not increase the sensitivity through an influence on the double-layer capacitance as in the present invention. Although metallic nanoparticles on electrodes may provide some increase in sensitivity by increasing the surface area of the electrodes, that is not the same mechanism as being exploited by the present invention. When an electric field is applied to a dielectric material, such as the dielectric nanoparticles (organic or inorganic) as in the present invention, there will be a redistribution of the charges in the material, which is often referred to as “polarization” of the material. Materials with higher dielectric constants will produce larger polarization of charges, which in turn result in higher capacitances. On the other hand, electric fields cannot penetrate into metallic or conducting materials, such as gold nanoparticles. Hence, there is no polarization and therefore no change in capacitive impedance. In short, dielectric nanoparticles can be capacitively coupled, whereas metal nanoparticles will not have this effect.

Embodiments of the present invention significantly increase the sensitivity by two or more orders of magnitude beyond the sensitivities of prior approaches through the use of nanoparticles that have a high dielectric constant, k. The principle behind the use of high k nanoparticles may be understood as follows. Capacitive and inductive components in a circuit create a phase difference between the applied voltage and current, resulting in an impedance circuit. By attaching an inductive or capacitive element to an analyte (or to a species that preferentially binds to the analyte), the profile of the Nyquist plot (which detects the impedance) will be altered. While it is difficult to incorporate an inductive element in this manner, capacitive elements can be introduced in form of high-k dielectric nanoparticles. In general, the larger the k value, the larger its effect will be on the impedance since capacitance is proportional to the k value. When the nanoparticle-analyte species binds to the electrodes, then the capacitive component will increase as well as the electron transfer resistance. No existing impedimetric sensor or impedance spectroscopy technique exploits high-k nanoparticles in this way to increase sensitivity. The dielectric constant k remains constant in most materials up to frequencies of several MHz. A large k value, or “high k” value is understood in the present context to mean a k value greater than 2 in this frequency range.

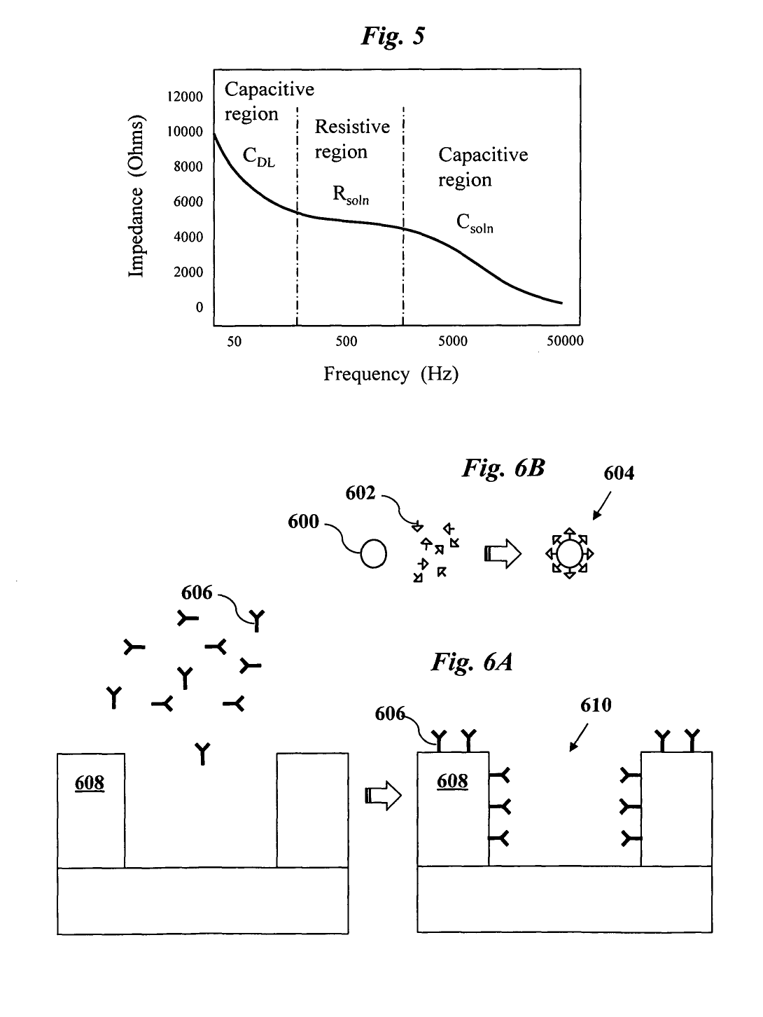

FIG. 5 is a graph of impedance magnitude as a function of frequency. At low frequency the impedance is characterized primarily by the double layer capacitance C_{DL}. In the mid-frequency range, the impedance is dominated by the resistance of the solution between the electrodes R_{Sol}, and at the high-frequency range, the capacitance of solution C_{Sol} between the electrodes dominates. Because immobilization of high-k dielectric nanoparticles on the electrode surfaces alters the double layer capacitance, a Nyquist plot of the impedance spectrum can be used for detection of specific analytes. The detection sensitivity will depend on the relative dielectric constant of the nanoparticles, e.g., silicon oxide nanoparticles have a dielectric constant (k) of about 3.9. However, the sensitivity can be enhanced by instead using TiO₂ nanoparticles (k=70-80). Other examples of high-k nanoparticle materials are HfO₂ (k=18-40), SrTiO₃ (k=60-200), and BaSrTiO₃ (k=120). In addition to these high-k inorganic nanoparticle materials, various high-k organic nanoparticle materials may also be used. For example, such materials include polystyrene (k=2.6) and polytetrafluoroethylene (k=2.1). Therefore, by choosing nanoparticle material of high dielectric constant, one can significantly amplify the Nyquist signal, hence the sensitivity of the impedimetric biosensor. Those skilled in the art will appreciate that many other inorganic and organic dielectric materials having high k values may also be used as the material for the nanoparticles. The nanoparticles may take various forms such as nanowires, nanotubes, nanorods, nanospheres, nanofibers, nanopowders, nanoclusters, nanobeads, or nanocrystals.

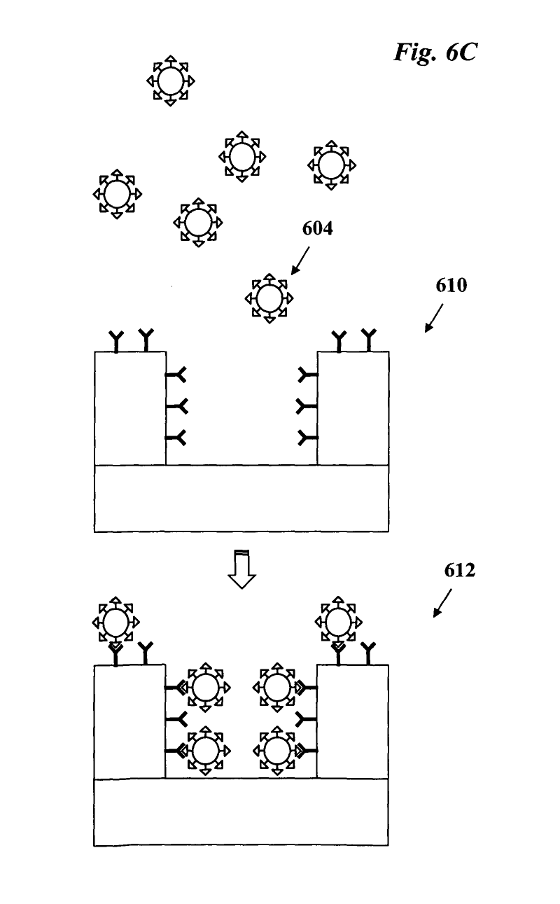

FIGS. 6A-C illustrate aspects of a method for impedimetric sensing of an analyte according to an embodiment of the invention. FIG. 6A shows the step of chemically functionalizing the surface of an interdigitated metal electrode 608 with a first species 606, such as an antibody, to produce a functionalized electrode 610. This functionalization process may involve an intermediate molecule to achieve the antibody attachment on the electrodes. The first species is selected such that it will preferentially attach to the analyte to be detected by the sensor. For example, the antibody is selected to complement an antigen to be detected.

FIG. 6B illustrates the step of chemically functionalizing a dielectric nanoparticle 600 with a second species 602, such as an antigen to be detected, to produce a nanoparticle-species compound 604. This step may be performed, for example, in a solution containing an unknown amount of the second species, or analyte. The first and second species may be biological species such as, for example, an antibody and complementary antigen or two complementary DNA strands.

The functionalized electrodes 610 are then exposed to the solution containing the nanoparticle-analyte compound 604, e.g., by flowing the solution over the electrodes. FIG. 6C illustrates the immobilizing or attaching of the functionalized nanoparticles 604 of FIG. 6B to the functionalized interdigitated electrode 610 of FIG. 6A as the solution is flowed over the electrodes, to produce functionalized electrodes with functionalized nanoparticles attached 612. After an incubation period, a buffer solution is used to purge the solution over the electrodes so that all the unattached nanoparticles are flushed out, so that the only nanoparticles in the device are those attached to the electrodes. The sensor is then ready to be characterized for impedance.

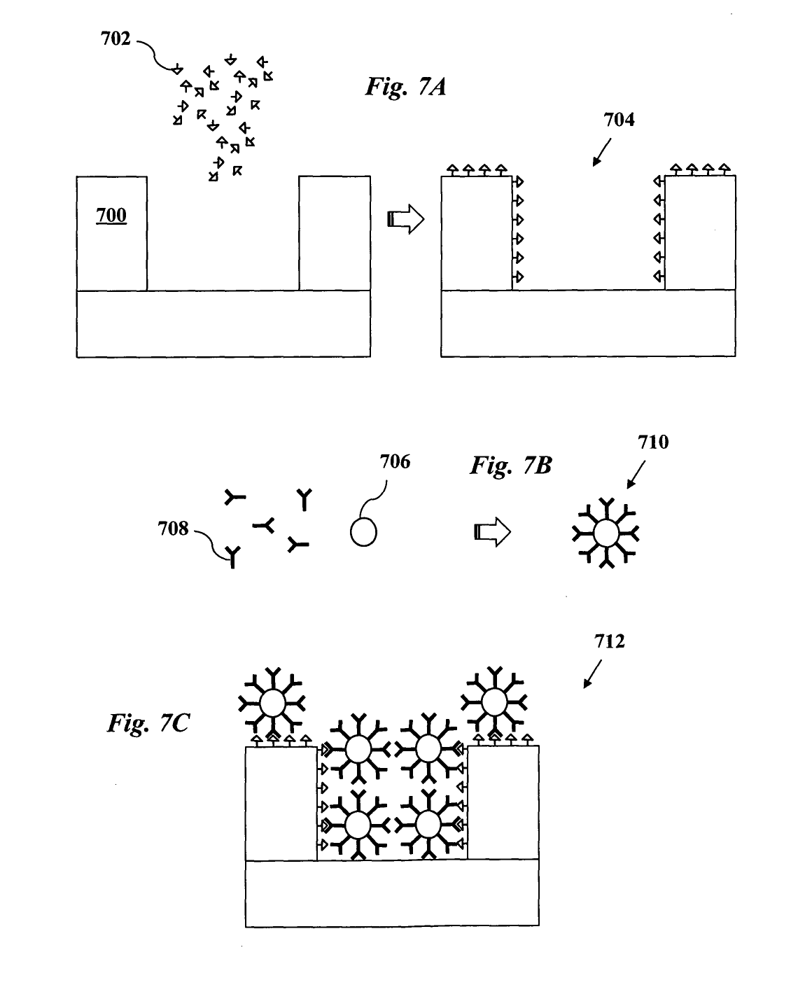

It should be noted that the analyte may be attached to the electrodes rather than to the nanoparticles, reversing the roles of the first species and second species. For example, FIG. 7A illustrates functionalizing an interdigitated electrode 700 with a first species 702, such as an antigen to be detected, e.g., by flowing a solution containing the antigen over the electrodes, to produce a functionalized electrode 704. After the electrode is functionalized, the solution with the first species is flushed out with a buffer. FIG. 7B shows functionalizing a dielectric nanoparticle 706 with a second species 708, such as an antibody, which may be performed in a second solution, to produce a functionalized nanoparticle 710. FIG. 7C is a schematic diagram illustrating the process of immobilizing or attaching the functionalized nanoparticles 710 of FIG. 7B to the functionalized interdigitated electrode 704 of FIG. 7A, e.g., as the second solution containing the functionalized nanoparticles is flowed over the electrodes, to produce functionalized electrodes with functionalized nanoparticles attached 712. The functionalization steps referred to above may be performed using standard techniques well known to those skilled in the art.

By way of example, in one embodiment, TiO₂ nanoparticles may be biofunctionalized with Protein-A as follows. The surface of nanoparticles can be decorated with desired chemically active functional groups such as amines and carboxyls. We have used silanization method to graft amine (—NH₂) groups on the surface of TiO₂ nanoparticles with APTES (amino propyl triethoxy silane). In brief, 1 g TiO₂ nanoparticles were mixed with 100 mL APTES solution (1% in ethanol). The nanoparticles were refluxed in APTES solution for eight hours. After refluxing, the nanoparticles were filtered and washed with excess ethanol. These nanoparticles were then kept at 200° C. for curing. This led to formation of multilayers of silanized surface through formation of Si—O—Si and Si—O—Ti bonds at the nanoparticle surface. The derivatized nanoparticles were further functionalized with Protein-A, a biomolecule that specifically binds to antibody molecules and much more strongly to IgG type antibodies. This was done by using a homobifunctional linker that links the amine groups of silane molecule at the nanoparticle surface to the amine groups of the Protein-A molecule. Glutaraldehyde is a highly effective and widely used homobifunctional linker molecule and was next introduced. Briefly, Protein-A solution was slowly added to the nanoparticle solution in presence of 0.1% glutaraldehyde solution in carbonate/bicarbonate buffer (0.05 M, pH 9.6). The solution was allowed to react at room temperature for 3 hours followed by conjugation reaction overnight at 4° C. The reaction was stopped using 10 μL/mL ethanolamine to quench any unreacted aldehyde groups. The nanoparticles were separated from the solution by centrifugation and the supernatant was discarded. Several washing steps were provided to ensure the complete removal of unbound or aggregated proteins. The non-specific binding sites were blocked using 3% BSA in phosphate buffered saline (PBS) for 3 hours. The nanoparticles were washed again and finally suspended in PBS containing 0.05% tween-20.

The immobilization of Transglutaminase (TG) antigen on the gold electrodes may be performed as follows. Transglutaminase (TG) is an antigen associated with celiac disease and is used for detection of anti-TG antibodies in serum of such patients. TG was immobilized on the gold electrodes as follows. The gold electrodes were cleaned with Piranha solution (1:4, peroxide: Conc. H₂SO₄) for 20 min. and washed thoroughly with deionized water. The electrodes were immersed in a solution of 50 mM Thioctic acid solution for 8 hours. Self-assembled monolayers were formed through gold-sulfur interactions and provided the surface with carboxyl groups (—COOH). The carboxyl groups were activated using 1-Ethyl-3-(3-dimethylaminopropyl)-carbodiimide (EDC)/N-hydroxysuccinimide (NHS) solution (100 mM each) in acetate buffer (0.05 M, pH 5.0) for 3 hours. The electrodes were washed with carbonate/bicarbonate buffer. After activation the electrodes were incubated with 50 μg/mL solution of TG in the same buffer overnight at room temperature. The reaction was stopped using 10 mM ethanolamine for 2 hours to quench any unreacted active carboxyl groups. Non-specific binding sites were blocked using 1% solution of Polyvinyl Pyrollidone in Phosphate Buffered Saline with Tween 20 (PBST). The chips were thoroughly washed and kept stored in PBST at 4° C. till further use.

The PrA-nanoparticle reagent may then be used for enhancing the binding of anti-TG antibody with TG antigen on the electrode surface. The electrodes were incubated with the solution containing anti-TG antibodies for 10 min. followed by a PBST wash and a 1:100 dilution of Pr-A-NP incubation for another 10 min. The impedance data was then collected after washing the electrodes with PBST.

According to one embodiment, the impedance values at a collection of distinct applied AC frequencies are measured in a circuit that includes the electrodes. An amount of the first species is then determined from the measured impedance values. Because the dielectric nanoparticles increase the double-layer capacitance, the sensitivity of determining the amount of the first species attached to the second species is improved. More specifically, impedance values may be measured by generating an AC signal at a predetermined frequency and applying the AC signal to the circuit. The frequency-dependent impedance of the circuit produced in response to the applied AC signal at the predetermined frequency is then measured by an impedance meter. This measurement is repeated with the predetermined frequency ranging over multiple distinct predetermined frequencies to obtain 100 to 1000 measured impedance values over a frequency range, e.g., 1 Hz to 100 kHz or more preferably 50 Hz to 50 kHz. The amount of the first species may then be determined by analyzing the measured impedance values to determine a double-layer capacitance at the chemically functionalized interdigitated electrodes, and comparing the capacitive impedance (i.e., impedance due to the double-layer capacitance), to calibrated impedance values to detect the amount of the first species attached to the second species. These impedance values can be calibrated against measurements using enzyme-linked immunosorbent assay (ELISA), which is an optical technique. The calibrated impedance values may be determined by performing similar measurements in the case where the amount of the analyte is known to be negligible or zero. Such similar measurements may be performed using the same electrodes at an earlier point in time prior to immobilization of the nanoparticles, using similar electrodes to which the nanoparticles have not been immobilized (e.g., because the electrodes were not functionalized or because the electrodes were not exposed to the analyte). Such similar electrodes may be electrodes in a common device or a distinct device. The analyzer may use common data analysis techniques such as Nyquist and Bode plots to detect a change in the measured values of the impedance of the double layers.

The techniques of the present invention have useful application including the detection of carcinoembryonic antigen, IgE antibody to a dust mite allergen protein, DNA complimentary target molecules, and sensing of cells. The methods may be implemented through the modification of existing interdigitated or planar electrode impedimetric sensor devices. A device for implementing the techniques may be realized in various forms including an all-electrical biochip, providing significantly increased sensitivity for detecting markers associated with various diseases. In one implementation of a chip, eight sets of electrodes are included for testing the same analyte sample. Six of the electrodes are selectively functionalized so that functionalized nanoparticles will attach to them, while two electrodes are left unfunctionalized so that no functionalized nanoparticles will attach to them. As a result, the two unfunctionalized electrodes may serve to provide reference signals or baseline signals for comparison to the other six electrodes. Such signals may be useful for detection purposes, e.g., to detect for non-specific binding and test for false positives.

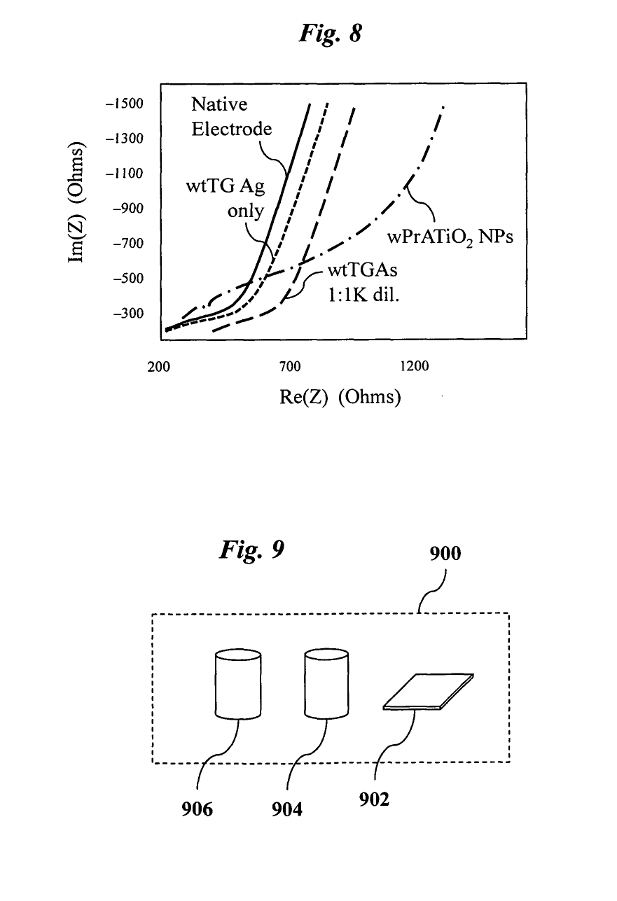

FIG. 8 shows a comparison of the Nyquist plots for different stages of biomolecular binding activity at the surface of electrodes. Specifically, plots of a native electrode are compared with that of an electrode with only wtTG Ag, and electrode with wtTGAs 1:1 K dil., and an electrode with wPrATiO2 nanoparticles. The native electrode is functionalized with tissue transglutaminase (tTG) antigen and blocked using 1% PVP. The immobilized antigen specifically captures the anti-tTG antibody from the antiserum (goat anti-TG used at 1:1 K dil. in PBST). Application of the PrA-TiO₂ nanoparticles is performed for signal enhancement. The charge transfer resistance increases with each successive layer formation on the electrode surface, and a significant change is observed after binding of tTG antibodies. The signal increases significantly after a signal enhancement step with PrA-TiO₂ nanoparticles binding, as indicated by the increasing diameter of the depressed semicircular part of the Nyquist plot.

In another embodiment of the invention, shown in FIG. 9, a kit 900 is provided that includes interdigitated electrodes (e.g., in the form of a chip 902) together with a solution 904 containing high-k nanoparticles. The electrodes in the kit may be functionalized with a first species (e.g., either the antibody or antigen), and the nanoparticles of certain concentration are provided in the kit with the complementary functionalization with a second species (e.g., either the antigen or antibody). In one use of such a kit, a patient blood sample at a certain concentration is added to either the nanoparticles or to the electrodes, and then the solution containing the nanoparticles are flowed over and allowed to attach to the electrodes in accordance with the techniques described earlier. In a real-time detection approach, the impedance is measured in a single step to determine either a yes/no result of the test. Alternatively, a two-step process where the blood sample would be mixed with the provided nanoparticle solution and flowed over the electrodes. This would be followed by flowing a buffer solution 906 (which may also be provided in the kit) so that only the attached nanoparticles would remain on the electrodes and all other access species swept out. The chip could then be tested.

1. A method for electrochemical impedance spectroscopy, the method comprising:
a) chemically functionalizing interdigitated electrodes with a first species;
b) chemically functionalizing nanoparticles with a second species that preferentially attaches to the first species; wherein the nanoparticles have dielectric constants greater than 2;
c) exposing the chemically functionalized interdigitated electrodes to a solution containing the chemically functionalized nanoparticles;
d) allowing the chemically functionalized nanoparticles in the solution to be immobilized on the chemically functionalized interdigitated electrodes through the attachment of the first species to the second species;
e) measuring impedance values of a circuit comprising the chemically functionalized interdigitated electrodes having the chemically functionalized nanoparticles attached, wherein the impedance values are measured at a plurality of distinct applied AC frequencies;
f) determining an amount of the first species from the measured impedance values;
wherein the nanoparticles increase the double-layer capacitance and improve the sensitivity of determining the amount of the first species attached to the second species.

2. The method of claim 1 wherein the measuring impedance values comprises:
g) generating an AC signal at a predetermined frequency;
h) applying the AC signal to the circuit comprising the chemically functionalized interdigitated electrodes;
i) measuring a frequency-dependent impedance of the circuit produced in response to the applied AC signal at the predetermined frequency;
j) repeating the generating, the applying, and the measuring such that the predetermined frequency ranges over multiple distinct predetermined frequencies to obtain measured impedance values over a frequency range.

3. The method of claim 1 wherein the determining the amount of the first species comprises:
analyzing the measured impedance values to determine a double-layer capacitive impedance at the chemically functionalized interdigitated electrodes;
comparing the double-layer capacitive impedance to calibrated impedance values to detect the amount of the first species attached to the second species.

4. The method of claim 1 wherein the nanoparticles comprise material with a dielectric constant (k value) greater than 10.

5. The method of claim 1 wherein the nanoparticles comprise an organic material.

6. The method of claim 1 wherein the frequency range is 1 Hz to 100 kHz.

7. The method of claim 1 wherein the frequency range is 50 Hz to 50 kHz.

8. The method of claim 1 wherein the first species is an antibody and the second species is an antigen complementary to the antibody.

9. The method of claim 1 wherein the second species is an antibody and the first species is an antigen complementary to the antibody.

10. The method of claim 1 wherein the first species is a first DNA strand and the second species is a second DNA that is complementary to the first DNA strand.

11. The method of claim 1 wherein the nanoparticles are nanostructures selected from the group consisting of nanowires, nanotubes, nanorods, nanospheres, nanofibers, nanopowders, nanoclusters, nanocrystals, and nanobeads.

12. A method for sensing an amount of an analyte in a solution, the method comprising:
a) binding nanoparticles to the analyte in the solution, wherein the nanoparticles have dielectric constants greater than 2;
b) chemically functionalizing interdigitated electrodes with a species that preferentially attaches to the analyte;
c) immobilizing the nanoparticle-analyte compound to the chemically functionalized electrodes in contact with the solution;
d) measuring impedance values of a circuit comprising the chemically functionalized electrodes having the immobilized nanoparticle-analyte compound attached, wherein the impedance values are measured at a plurality of distinct applied AC frequencies;
e) determining the amount of the analyte from the measured impedance values.

13. The method of claim 12 wherein the nanoparticles have dielectric constants greater than 10.

14. A method for sensing an amount of an analyte in a solution, the method comprising:
a) binding the analyte to interdigitated electrodes in contact with the solution;
b) chemically functionalizing nanoparticles with a species that preferentially attaches to the analyte, wherein the nanoparticles have dielectric constants greater than 2;
c) immobilizing the chemically functionalized nanoparticles to the analyte bound to the interdigitated electrodes;
d) measuring impedance values of a circuit comprising the interdigitated electrodes having the chemically functionalized nanoparticles immobilized on the bound analyte, wherein the impedance values are measured at a plurality of distinct applied AC frequencies;
e) determining the amount of the analyte from the measured impedance values.

15. The method of claim 14 wherein the nanoparticles have dielectric constants greater than 10.

16. The method of claim 14 wherein the species is a biological species.

17. A kit comprising:
a) a solution containing nanoparticles, wherein the nanoparticles have dielectric constants greater than 2, and wherein the nanoparticles are chemically functionalized with a first species; and
b) interdigitated electrodes that are capable of chemically functionalized with a second species that preferentially attaches to the first species.

18. The kit of claim 17 further comprising a buffer solution.

19. A kit comprising:
a) a solution containing nanoparticles, wherein the nanoparticles have dielectric constants greater than 2, and wherein the nanoparticles are capable of being chemically functionalized with a first species; and
b) interdigitated electrodes that are chemically functionalized with a second species that preferentially attaches to the first species.

20. The kit of claim 19 further comprising a buffer solution.
